(19) United States
(12) Patent Application Publication
(10) Pub. No.: US 2025/0177613 A1
(43) Pub. Date: Jun. 5, 2025

(54) LASER-APPLIED MARKINGS FOR MEDICAL DEVICES FOR IMPROVED SONOGRAPHIC AND RADIOLOGICAL IMAGING

(71) Applicant: THÜRINGISCHES INSTITUTE FÜR TEXTILE- UND KUNSTOSTOFF-FORSCHUNG E.V., Rudolstadt (DE)

(72) Inventors: Janine BAUER, Rudolstadt (DE); Holger GUNKEL, Rudolstadt (DE); Stefan HEINEMANN, Rudolstadt (DE)

(73) Assignee: THUERINGISCHES INSTITUTE FUER TEXTILE- UND KUNSTOSTOFF-FORSCHUNG E.V., Rudolstadt (DE)

(21) Appl. No.: 18/844,998

(22) PCT Filed: Feb. 27, 2023
(86) PCT No.: PCT/EP2023/054860

§ 371 Date: Sep. 9, 2024

(30) Foreign Application Priority Data
Mar. 9, 2022 (DE) 10 2022 105 492.6

(51) Int. Cl. A61L 29/08

(52) U.S. Cl. CPC A61L 29/085

(57) ABSTRACT

The invention relates to a method for creating visibility-enhancing markers which can be applied onto a wide variety of materials with a very high degree of geometric variability and which can be used both for radiological as well as for sonographic applications. According to the invention, this is achieved substantially by way of novel markings created by means of a polyurethane coating into which a particle film with functional properties and high particle density is embedded. By using a laser, only targeted coating regions in selected areas are dried and chemically cross-linked. Exposed areas are created by subsequently rinsing off non-irradiated coating material. The markings can thus be created in any geometric shape.

The invention relates to a method for producing medical devices having markings visible for imaging methods. The invention further relates to medical devices producible by the method.

The in vitro visibility of medical instruments and implants for therapeutic and diagnostic purposes by means of ultrasound (US) and radiography methods is of high clinical relevance.

For optimal placement near the desired site of action and to avoid complications, medical devices in their entirety should be readily depictable during application until inspection of the final position.

Internal patient structures and/or objects within a patient are visualized by using mainly radiological and sonographic techniques.

In particular, the present invention relates to catheters. Catheters are usually used for drainage, for flushing, for administering diagnostic or therapeutic drugs, for biopsy or for establishing a passage within hollow organs and for various other procedures. Such catheters mainly comprise a base body made of a polymeric material.

Since the plastics materials used have very poor ultrasound visibility (echogenicity) and X-ray visibility (radiographic contrast), the placement of an invasive catheter and inspection of the position thereof is, however, found to be difficult. Catheters currently available on the market are reliably visualizable by ultrasound only at depths of a few millimeters below the skin surface.

Limiting factors in identification are the difference in the material constants (acoustic impedance and X-ray absorption) between body tissue and catheter, and the geometric dimensions of the catheter.

The invention comprises a method for producing visibility-enhancing markers which can be applied with very high geometric variability to a variety of materials and which can be used for both radiological and sonographic applications.

In order to detect the position of a catheter within a patient in its entire length by means of radiodiagnostics, the entire catheter material or strips thereof is/are filled with radiopaque substances such as barium sulfate. For identification of individual regions of a catheter, for example the distal end, radiopaque markers are commonly used.

It is known to implement the markers in the form of a coating, a band or an inlay. For example, rings of solid radiopaque metal are attached on the catheter tube. Since the solid metal band is relatively inflexible compared to the catheter shaft material, local stiffnesses and undesirable discontinuities are produced on the catheter. Bending and/or torsional stress can cause failure of the material composite and hence loss of the marker. Furthermore, metallic markers are relatively expensive to produce and difficult to reliably attach to an underlying device.

Many of the problems associated with the use of metallic markers are overcome by replacing the rigid precious metal tube with a polymer filled or doped with a suitable radiopaque material.

As described in U.S. Pat. No. 6,540,721 and WO 2005/030284, such markers are produced by mixing a polymer resin with a pulverulent, radiopaque material such as elemental tungsten followed by extrusion of the composition. Because of the method and because of the high differences in density between the metal and the polymer, only low volume fractions of the metal can be introduced into the compound. Excessively large wall thicknesses are therefore required to achieve sufficient radiographic contrast. The attachment of the markers on the instrument produces protrusions, and the profile is altered in an undesirable manner. In many cases, external restrictions on dimensions prevent the use of devices having such markings.

The same applies to the method of the application DE 10 020 739 and WO 2021/029348, wherein wound high-contrast tubes or wires allow better identification of the position and the deformation of a surgical instrument. The freedom of design as regards geometric radiograph patterns which can be produced by means of these methods is also very limited here.

Ultrasound-visible instruments and markings require the use of other methods. The approaches are primarily based on the principle that gaseous substances have an enormous difference in acoustic impedance in relation to solids and also in relation to human tissue. Taking advantage of the high impedance difference at the gas/solid interface, what are proposed in many cases are substrates or coatings which, for example, comprise gas pockets, voids, pores, gas-containing channels or microscopic surface structures for holding surface air inclusions.

WO 98/18387 discloses medical instruments, such as needles, of which a portion of the surface has been covered with a material, such as epoxy resin, which has been filled with reactive substances as bubble generator. Upon contact with a liquid, which can occur upon insertion into a tissue, the substances, such as sodium hydrogencarbonate and citric acid, react with release of gas and form a large number of mobile bubbles. Open-pore structures bring about rough surfaces and can bring about the desired contrast just briefly, since the gas bubbles gradually break up and the surface is wetted with liquid.

Closed pore structures of defined cell geometry and high homogeneity can be implemented as syntactic foams by embedding hollow spheres. DE 20 2009 001 974 U1 discloses full-surface coatings containing voids which are produced by embedding hollow microspheres made of vinylidene chloride, which for their part can be filled with gas, such as isobutane.

Further known are catheters which are characterized by a multilayer construction which is produced by extrusion. Modification of individual layers can improve the echogenic properties. EP 1 462 056 relates to a catheter consisting of at least two layers, of which the outer layer has a greater layer thickness than the inner layer and gas bubbles have been dispersed into the outer layer. Layers produced in this way have the disadvantage that they are present on the entire length of the extruded part and are thus also present in regions in which they are rather undesired. It is not possible to produce patterned markings for better differentiation from endogenous structures and from method-related noise in the ultrasound image. The physical properties of the device are influenced greatly. For example, the property of transparency that is often important for catheters is lost.

The solution proposed in EP 3 738 543 to implement a catheter in multilayer form and to use laser induction to foam a region to be marked offers extensive possibilities for the graphic design of echogenic markings, but is dependent on specifically implemented catheter shafts which have been extruded in multilayer form and filled with laser absorbers. It is not possible to furnish conventional catheter tubes with such echogenic markings.

US 2014/0221828 A1 discloses medical devices having checkered echogenic patterns which are produced by casting or printing of a metal film or by gas-filled plastics structures. In the form shown, the laser treatment recommended for the plastics structuring has the disadvantage that ablation and bubble formation results in the formation of depressions and elevations on the surface. The material changes caused by laser beams are known to become effective especially in the near-surface region and to decrease with increasing layer depth. No solution is shown as to how the effect of the laser beam can be restricted to the interior of the catheter wall and how the formation of surface unevenesses is avoided.

It is an object of the invention to create visibility-enhancing markings on medical devices, in particular catheters, which can be used in both radiological and sonographic imaging and can be placed precisely and easily at any desired site with very high geometric variability.

The instruments are to keep their transparency and to be provided with visible markings only in the regions which are of interest for detectability and possible subsequent manipulation. By designing the markings in the form of graphics, scales and patterns, the detection of displacements, twists, bends, shrinkage or expansion of the apparatus and the identification of functional regions are to be improved.

Furthermore, a method is to be provided that allows the simple and economic production of the markings on medical devices.

To achieve the object, the invention essentially envisages producing the novel markings by a polyurethane coating in which a particle film having functional properties has been embedded at high occupation density.

By using a laser, only specific site-selective regions of the coating are dried and chemically crosslinked. The markings are made available by subsequently rinsing off nonirradiated coating material. The markings can thus be implemented in any geometric form.

The particles are selected depending on the desired functionality. For example, hollow glass microspheres are used for sonographic applications and spherical tantalum particles are used for X-ray visibility.

By applying an additional particle-free layer as a cover (5), optimal particle adherence and surface smoothness can be achieved.

For the coatings, preference is given to using polyurethane systems. Polyurethanes formed of are by polyaddition bifunctional or higher functional alkanols and isocyanates. They are available from a variety of sources and can be applied very efficiently to a variety of substrates, such as metals, textiles, ceramics and heat-sensitive plastics. Preference is given to marking medical devices made of plastic. Available on the market for this purpose are coating systems of modular design that allow high flexibility in formulation design and in application properties. In many cases, polyurethanes also ensure good biocompatibility, thus allowing use for medical purposes.

Use is made of solvent-containing systems or aqueous dispersions and emulsions. Water-based compositions have improved biocompatibility and biostability and they are more environmentally friendly. By dispensing with organic solvents, there is often also better compatibility in relation to the substrate. To quicken the polymerization reaction, use can be made of the catalysts known in polyurethane chemistry. In general, the amount of catalyst required is very low and is in the order of magnitude of 100 ppm or below. Preference is given to synthesis without a catalyst.

Particular preference is given to the use of so-called stoving systems which only chemically react and cure under increased supply of heat. The temperature is in the range between 10° and 200° C. The time required for sufficient crosslinking is in the range between 2 and 100 min and can be shortened by higher temperatures.

The polyurethane layers are applied to well cleaned and possibly activated substrates by conventional methods such as printing, spraying, doctor blading and dipping. As a result of specific dilution or the use of thickeners, layer thicknesses in the range from 500 nm up to a few 100 μm can be achieved, preference being given to 5 to 50 μm in the context of the present invention.

Alternatively, also suitable are other known coatings which can be dried and cured by heat action or UV rays and exhibit biocompatible, nontoxic, hypoallergenic and stable behavior for medical use.

According to the invention, such a coating may also be selected from the group of sol-gel systems, acrylates, melamine, polyester.

The tackiness of the coating material directly after application is taken advantage of for adherence of functional particles. To this end, the coated part is sprayed with particles or dipped into a static or fluidized powder, such that particles accumulate as a film on the coating. The definition of a film here is a virtually blanket single layer of particles having an average height only negligibly above the average height of the particles.

To this end, preference is given to using a fluidizing tank in which air is supplied from below through minute openings and said air whirls up the powder, thereby causing it to freely circulate. In the case of a low layer thickness of the particles on the polyurethane layer high occupation density, relatively fine and spherical geometries are found to be advantageous here.

The coating structure thus formed is then dried and cured by laser-induced heat input. A computer-controlled optical system allows rapidly deflectable laser pulses of the desired output to act specifically at the sites to be heated. The introduction of heat is exactly defined thermally and geometrically. The programmed movement of the laser beam allows high-precision curing of not only imaging regions of relatively large extent, but also labels, patterns, markings and the like of small surface area. The marking formed may be in the form of a single piece or else comprise multiple parts which can be connected to one another. With the aid of turning apparatuses, circumferential markings are also possible.

For this purpose, especially also for use on devices made of plastic, preference is given to using cost-effective diode-pumped solid-state lasers and fiber lasers in the wavelength region of 1064 nm (NIR), as similarly also used for marking and labeling. The form of the marking is programmed with the aid of labeling software.

Furthermore, the use of an excimer laser, for example by way of a masking technique, is also possible. However, other conventional laser types having a wavelength in a region of high absorption by the absorber materials used, for example CO₂ laser and other dye laser, can also achieve the desired results. The output of the lasers used and the setting of various laser parameters depend on the particular application and can be readily determined by a person skilled in the art on a case-by-case basis.

The laser energy is absorbed in the substrate in the case of metallic apparatuses, and so the coating material is heated indirectly. Unfilled catheter plastics such as TPU, PVC and silicone and the polyurethane coating itself are, however, laser-transmissive at wavelengths of near-ultraviolet to near-infrared light, i.e., they show no interaction with the laser radiation. It has been found that metallic particles actually applied to the coating for the purpose of radiographic contrast can also bring about very good laser absorption and hence heat input into the coating. The adherent metallic particles make it possible for the polyurethane reaction mixture without additional added laser additives to be cured by laser treatment even on laser-transparent heat-sensitive materials. The specific transfer of heat from the solid particle into the polyurethane layer achieves very strong attachment of the particles at the sites of contact. The necessary reaction time is ensured by guiding the laser beam repeatedly across the area to be treated.

The markings are made actually available by rinsing off the nonirradiated and hence non-crosslinked coating material. The rinsing liquid is selected depending on the coating system applied and the substrate. In the case of aqueous composites, preference is given to using water for rinsing. If organic solvents are necessary, an action by solvent on the substrate and the crosslinked coating must be ruled out. Suitable solvents may be ketones, for example acetone, butanone, methyl ethyl ketone and methyl isobutyl ketone, cyclic or aromatic hydrocarbons, for example xylene, toluene and cyclohexane, or esters, for example butyl acetate, ethyl acetate and methoxypropyl acetate, or mixtures of the aforementioned substances.

The reliability of particle adherence can be further increased and surface roughness can be reduced by application of a further polyurethane layer which is cured conventionally or by laser treatment. This also achieves an insulation which prevents undesirable electrochemical processes in the blood or other body fluids between the metal particles and the instrument. Furthermore, it has been found to be advantageous to subject the medical device having the applied markings to a temperature treatment in a heat cabinet which ensures that the polyurethane coating has fully reacted to completion and that the solvent has completely evaporated.

The required functionality of the markings according to the invention is ensured by the embedded particles. The radiopaque powder materials suitable for imaging in radiodiagnostics include metallurgical materials having a high atomic number such as platinum, tantalum, iridium, tungsten, rhenium, gold and alloys of these metals. To produce markings on metallic apparatuses, metal compounds such as tungsten carbide, tungsten boride, barium sulfate and bismuth oxychloride may also be used.

The shape of the particles is not crucial, though it should preferably be spherical. Suitable particle sizes are in the range from 1 to 50 μm, and diameters of 5-10 μm are especially preferred. Smaller particles produce a thinner coating which is less detrimental to the properties of the medical device. In the case of nonspherical particles, the sizes indicated relate to the equivalent sphere diameter, i.e., the diameter of a sphere having the same volume as the particle.

One of the major advantages of the method according to the invention is that the particles are embedded at a very high fraction in the total volume of the coating. The functional volume fraction may be 40-75% and is preferably realized in the range from 50 to 70%.

In accordance with the prior art, hollow microbodies can be embedded into the marking composite for the function of ultrasound visibility. Especially suitable are hollow glass and ceramic spheres and polymeric microspheres.

Alternatively, expandable microspheres are used. The particles are microscopically small spheres having a thermoplastic outer skin and a filling composed of a condensed gas which expands on heating. Besides the use of already expanded hollow spheres, there is the possibility of expanding nonexpanded types as part of laser treatment. The fraction of functional volume in the coating can thus be increased yet further.

Compared to rigid hollow spheres, polymeric elastic microbubbles can allow better ultrasound visibility. Not only do they provide an acoustic impedance drastically different from human tissue, but they also act as resonators for ultrasound waves and thus increase the backscattering effect.

Preferably, echogenic microparticles having a diameter between 1 and 50 μm are used.

Because of the very low laser absorption of hollow microbodies, the use thereof in the context of the method according to the invention is, however, restricted to apparatuses comprising metallic materials. Surprisingly, it has been found that a coating structure having embedded metal particles also produces very good echogenic behavior. It has to be assumed that the method gives rise to voids in the coating composite that are completely not filled with polymer, even by the final application of top layer. The second polyurethane layer leads to a closed-pore structure, thus preserving the voids even in the case of contact with body fluids and other liquids. The gas inclusions in the coatings cause strong reflection of the sound waves, with the result that they are imaged much more brightly in the ultrasound image compared to the surrounding substances.

This method makes it possible to create markings on metallic and nonmetallic apparatuses which allow improved imaging with both radiological and sonographic diagnostic methods. The studies on the examples listed show that, despite the bifunctional effect of the markings, both methods achieve high image quality with perfect contrasts and with a high level of detail. The markings are distinguished by high wipe resistance and scratch resistance and are stable in subsequent sterilization processes.

The claimed method is thus especially suitable for marking catheters where only a few technical solutions were previously available for improving detectability. The preferred patterning of the markings allows simple differentiation from endogenous structures and permits easy detection of displacements, bends or twists. Furthermore, there is the option to scale regions of particular interest by means of patterns and to highlight them for subsequent manipulation of the catheter.

Besides the use on catheters, the method may also be applied to other medical devices which are inserted or implanted into an animal or human body. A medical device according to the present invention is therefore preferably selected from the group consisting of catheters, needles, stents, cannulas, tracheotomes, endoscopes, dilators, tubes, introducers, markers, stylets, snares, angioplasty devices, fiducial markers, trocars and tweezers.

In the following, the invention will be elucidated on the basis of an exemplary embodiment. Further details, advantages and features of the invention will become immediately apparent from the claims.

The drawings show in

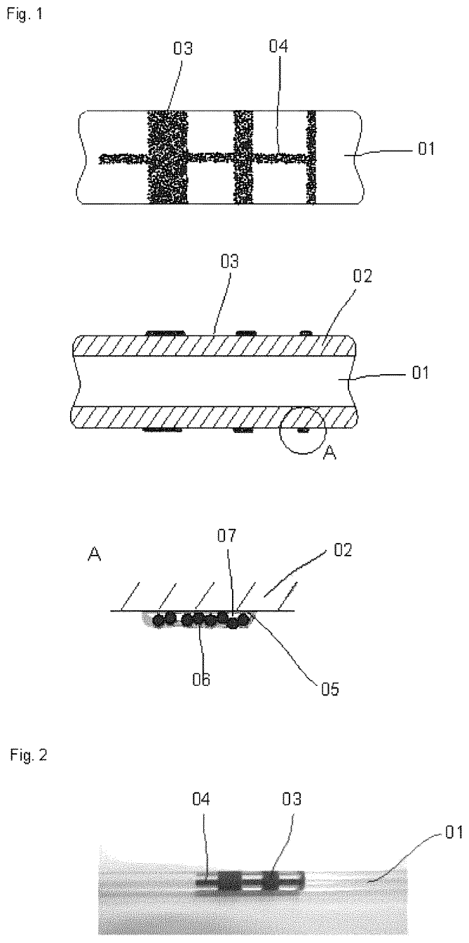

FIG. 1 a schematic illustration of markings on a catheter that are produced according to Example 1,

FIG. 2 a photograph of a catheter furnished with markings according to Example 1,

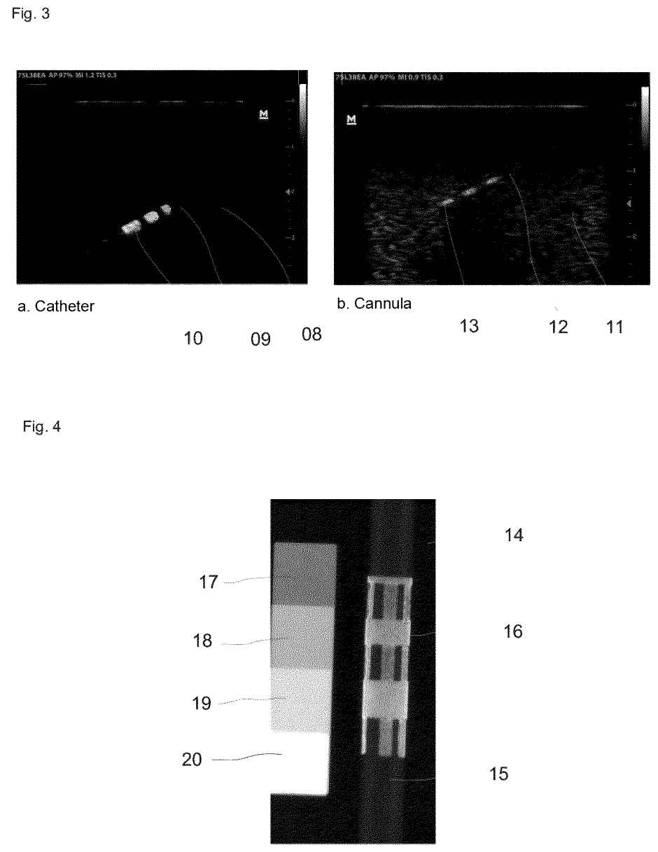

FIG. 3 ultrasound images of the markings produced according to Examples 1 and 2
(a) measured on a catheter and in water
(b) measured on a cannula and on an ultrasound phantom

FIG. 4 a radiograph of a catheter produced according to Example 1.

FIG. 1 shows various views of the structure in principle of a catheter 01 provided with markings 03, 04. By way of example, the catheter is implemented with 3 annular markings 03 which are arranged circumferentially with differing width, and with 3 markings which connect the rings as longitudinally arranged bridges 04.

The markings lie on the catheter wall 02 and are characterized by a structure in which a film of closely packed metal particles 07 is embedded between two polyurethane layers 05, 06. The spaces between the particles are not completely filled with polymer, meaning the presence here of air-containing voids 08. The voids are outwardly closed by the top layer 06. The radiopaque spherical metal particles 07 have an average size of 10 μm and occupy a fraction of 50-70% of the volume of the marking. The total thickness of the coating is, at approx. 15 μm, only a little above the diameter of the individual particles.

The markings shown in FIG. 2 were produced on a TPU catheter having an outer diameter of 3 mm and have the structure according to FIG. 1.

Markings produced according to the invention stand out in the radiograph (FIG. 3) and ultrasound image (FIG. 4) with very high brightness contrasts and good contour definitions against the black background and against the untreated regions of the instruments.

The following examples serve to illustrate the invention. In said examples, percentages are to be understood to mean percentages by weight, unless indicated otherwise or unless apparent from the context.

### Materials Used

Unless stated otherwise, all materials were purchased from CSC JÄKLECHEMIE GmbH & Co. KG.

Elastollan® 1180 A10 FC is a TPU from BASF that has a Shore A hardness of 80, a strength of 45 MPa and an elongation at break of 650%.

Desmophen® T1777 is a polyol component for production of polyurethane stoving coatings with blocked aliphatic polyisocyanates from Covestro.

Desmodur® BL 3475 is a diethyl malonate-blocked, aliphatic polyisocyanate based on isophorone diisocyanate (IPDI) and hexamethylene diisocyanate (HDI) from Covestro.

Bayhytherm® 3246 is a water-thinnable, aliphatic, self-crosslinking stoving urethane resin from Covestro.

### Methods

The ultrasound visibility studies were performed in B mode with a DP-50 ultrasound diagnostic machine from Mindray and a linear sonic head at a sound frequency of 8 MHz. For the ultrasound images, the various devices were positioned in a 45° position in relation to the sound direction, both in a water bath and in an ultrasound phantom having tissue-like properties. To assess the contrast of the marking relative to unmarked regions, image processing software was used to average the grayscale spectra (histograms) of the individual regions of the ultrasound image and to compare them with one another, with 100% black corresponding to a value of 0 and 100% white corresponding to a value of 255.

Radiographic imaging was studied with an intraoral radiography system from Trophy-Radiologie GmbH with digital sensor technology. As comparative standard, the measurement included an aluminum strip with graduated thicknesses of 1, 2, 3 and 4 mm. The exposure setting of the radiography machine was chosen such that the 4 levels were imaged with high contrast differences. (70 kV, 10 mA, 0.5 seconds, sensor size 18×24 cm).

### Example 1

This example describes the production of a marking according to the invention on a catheter.

A tube extrusion system is used to produce a catheter having an outer diameter of 3 mm and a layer thickness of 0.5 mm. The tube material is a TPU of the type Elastollan® 1180 A10 FC.

A coating material is prepared by adding the following ingredients to a beaker and mixing them:
Desmophen® T1777: 34.8%
Desmodur® BL 3475: 27.9%
1-Methoxy-2-propyl acetate: 37.2%.

The catheter is closed at one end by a stopper, dipped into the coating solution and then pulled out very slowly at a rate of 1 mm/s. The excess solution can drip off and the coated tube is dried at room temperature for 2 minutes.

In a fluidizing tank, tantalum powder having an average particle size of 8 μm is whirled up by compressed air. The catheter is dipped into the fluidized bed while turning and remains there until sufficient particles are adhering to the tacky coating. After a further drying time of 10 minutes, the tube is irradiated with a pulsed 10 watts Yb fiber laser from FOBA. By choosing suitable laser parameters (laser output: 20%, speed of travel: 10 mm/s, pulse frequency: 20 kHz), an increase in temperature is brought about at the irradiated sites 03, 04 that leads to crosslinking and curing of the coating. The movement of the laser beam to form the contour and to fill the areas is repeated multiple times, such that the local increase in temperature is maintained for a period of 20 min.

The circumferential ring markings 03 were programmed as rectangles by the laser labeling software and realized by axial rotation of the tube during the laser treatment. The bridges 04 were produced on a stationary tube by repeated lasering after every 120° turn of the tube. After completion of the laser treatment, the coated part of the catheter is dipped into 1-methoxy-2-propyl acetate and the noncured coating material rinsed off by agitation.

After a drying time of 10 min, the top layer is formed by repeating the procedure comprising dip coating, laser treatment and rinsing. The application of particles is omitted in this step. The result is a marking as shown in FIG. 2.

FIG. 3a shows the ultrasound images of the catheter 01 dipped in water at a 45° position in relation to the transmitted ultrasound. The imaging of the marked catheter in a radiograph can be seen in FIG. 4. In order to allow a comparison with customary practice, the image included a standard (aluminum plate with the thickness gradation of 1, 2, 3 and 4 mm).

The following tables give an overview of the average brightness values of the various image regions, as determined from a grayscale histogram.

| | Average brightness |
|---|---|
| Region of the image (FIG. 3a) | values, ultrasound image |
| Background 08 | 1 |
| Unmarked catheter region 09 | 10 |
| Marking on catheter 10 | 205 |

| | Average brightness |
|---|---|
| Region of the image (FIG. 4) | values, radiograph |
| Background 14 | 11 |
| Unmarked catheter region 15 | 28 |
| Marking on catheter 16 | 168 |
| Aluminum standard, 1 mm 17 | 119 |
| Aluminum standard, 2 mm 18 | 177 |
| Aluminum standard, 3 mm 19 | 218 |
| Aluminum standard, 4 mm 20 | 249 |

The high level of functionality of the imaging markings is demonstrated by both subjective visual inspection and digital analysis of the images. The marking has very good radiopacity corresponding to approximately a 2 mm thick aluminum plate.

### Example 2

This example describes the production of a marking according to the invention on a 22 G disposable cannula (outer diameter: 0.7 mm) made of stainless chromium-nickel steel.

In contrast to Example 1, the coating material is prepared from the following components:
Bayhytherm® 3246: 90%
Distilled water: 10%.

The particles used are hollow glass microspheres of the type 3M™ Glass Bubbles K37 having an average particle size of 40 μm. The marking is in the form of 3 circumferential rings having a width of 3 mm.

The sonographic studies were performed on a commercial ultrasound phantom of tissue-imitating material. FIG. 3b shows an ultrasound image of the 0.7 mm thin needle at an ultrasound frequency of 8 MHz at a depth of 1 to 2 cm.

The following table gives an overview of the average brightness values of the various image regions in the ultrasound image, as calculated from a grayscale histogram (FIG. 3b).

Despite the small diameter of the needles, the markings are distinguished by very good ultrasound visibility.

| | Average brightness |
|---|---|
| Region of the image (FIG. 3b) | values, ultrasound image |
| Background 11 | 8.5 |
| Unmarked catheter region 12 | 12.3 |
| Marking on catheter 13 | 143.1 |

### LIST OF REFERENCE SIGNS

01—Catheter
02—Catheter wall
03—Ring marking
04—Bridge marking
05—PU base layer
06—PU top layer
07—Particle film

1. A method for producing a medical device having markings visible for imaging methods comprising the following steps:
a. applying a thin layer of a liquid thermally curable polyurethane coating to the device to be provided with markings;
b. adhering a film of hollow microspheres or of metal particles onto the polyurethane coating applied in step a);
c. laser curing the coating from step b) at defined sites that form patterns, with the patterns selected such that only a portion of the coating material cures;
d. rinsing off non-crosslinked coating using a suitable solvent; and
e. applying and curing a top layer.

2. The method as claimed in claim 1, wherein the metal particles are 1 to 50 μm in size.

3. The method as claimed in claim 1, wherein the metal particles have a density greater than 4 g/cm³ and predominantly consist of metals having an atomic number greater than 21 or compounds thereof.

4. The method as claimed in claim 1, wherein the hollow microspheres have a particle size of 10 to 80 μm.

5. The method as claimed in claim 1, wherein the hollow microspheres are hollow glass and ceramic spheres or polymeric microspheres.

6. The method as claimed in claim 1, wherein the particles have a volume fraction in the coating is of 40 to 75%.

7. The method as claimed in claim 1, wherein the markings are applied in the form of symbols, shapes, graphics, scales or patterns.

8. The method as claimed in claim 1, wherein the step of adhering the film by comprises dipping the device of step a) into a fluidizing tank containing a fluidizing powder or spraying the device of step a) or doctor blading the device of step a).

9. The method as claimed in claim 1, wherein the laser curing of the coating comprises irradiating the coating with laser light having a wavelength in the near-UV region and/or visible region and/or near-IR region.

10. A medical device having marking for imaging methods, wherein said device has been produced by a method as claimed in claim 1.

11. The medical device as claimed in claim 10, wherein said device is a catheter, needle, stent, cannula, tracheotome, endoscope, dilator, tube, introducer, marker, stylet, snare, angioplasty device, trocar or tweezer.

12. The method as claimed in claim 2, wherein the metal particles are 1 to 10 μm in size.

13. The method as claimed in claim 3, wherein the metal is platinum, tantalum, iridium, tungsten, rhenium, gold or alloys of these metals and the metal compounds are tungsten carbide, tungsten boride, barium sulfate or bismuth oxychloride.

14. The method as claimed in claim 4, wherein the hollow microspheres have a particle size of 30 to 50 μm.

15. The method as claimed in claim 6, wherein the volume fraction of particles in the coating is 50 to 70%.
